(19) United States
(12) Patent Application Publication
(10) Pub. No.: US 2025/0144051 A1
(43) Pub. Date: May 8, 2025

(54) Colchicine for Treating Connective Tissue Disorders

(71) Applicant: Khan Sciences, Inc., Fairfax Station, VA (US)

(72) Inventors: Ejaz Khan, Fairfax Station, VA (US); Shamayal Khan, Fairfax Station, VA (US)

(21) Appl. No.: 18/939,296

(22) Filed: Nov. 6, 2024

Related U.S. Application Data
(60) Provisional application No. 63/596,634, filed on Nov. 7, 2023.
(60) Provisional application No. 63/616,524, filed on Dec. 30, 2023.

(51) Int. Cl. A61K 31/165; A61K 31/4415; A61P 19/04

(52) U.S. Cl. CPC A61K 31/165; A61K 31/4415; A61P 19/04

(57) ABSTRACT

A method of treating a connective tissue disorder in a patient. The method comprises orally administering colchicine in a therapeutically effective dose amount. This treatment could suppress the pathologic inflammation, fibrosis, or angiogenesis associated with such connective tissue disorders. As an example, the connective tissue disorder could be tendinopathy (such as carpal tunnel syndrome). The treatment may have various beneficial effects at the target connective tissue, such as reduced fibrosis, increased collagen turnover, increased extracellular matrix turnover, reduced inflammation, reduced angiogenesis, etc.

### TECHNICAL FIELD

This invention relates to the medication colchicine.

### BACKGROUND

Repetitive strain injury (also sometimes called repetitive stress injury) is a musculoskeletal condition resulting from repetitive overuse and can occur in many different body locations. Two common types of repetitive strain injuries are tendonitis and carpal tunnel syndrome. Tendonitis is inflammation in tendons with resulting pain and swelling. For example, in carpal tunnel syndrome, repetitive use of fingers (e.g. in a job or activity) causes inflammation of the transverse carpal ligament (carpal tunnel) or the flexor tendons in the wrist. This results in compression on the median nerve, which causes pain, numbness, tingling, and weakness in the hand and wrist. These injuries often require treatment by rehabilitation, physical therapy, NSAIDs (nonsteroidal anti-inflammatory drugs), and sometimes even surgery. However, these approaches do not fully address the underlying fibrosis, inflammation, abnormal healing, or angiogenesis that contributes to the progression of connective tissue disorders. Thus, there is a need for alternative treatments for carpal tunnel syndrome and other connective tissue disorders.

### SUMMARY

TREATMENT METHOD. In one aspect, this invention is a method of treating a connective tissue disorder in a patient. This treatment could suppress the pathologic inflammation, fibrosis, or angiogenesis associated with such connective tissue disorders. The treatment may be for any suitable type of connective tissue such as muscles, tendons, tendon sheaths, subsynovial connective tissue (SSCT), fascia, or ligaments. The term “connective tissue” as used herein excludes joints and blood vessels. As such, the term “connective tissue disorder” excludes joint arthritis and conditions involving blood vessel inflammation (as examples). The treatment may be particularly suitable for connective tissue composed of dense collagen fibers, such as tendons or ligaments. In some cases, the target treatment site is in the upper extremity (e.g. arms, wrists, hands, fingers).

The method comprises administering a therapeutically effective dosage amount of colchicine to the patient for treating the connective tissue disorder. Any suitable therapeutic route could be used, including oral, intravenous, intramuscular, transdermal, etc. In some embodiments, the route of administration is oral and the dose amount is in the range of 0.2-1.9 mg; and in some cases, 0.5-1.5 mg. A single dose or multiple (two or more) doses may be given daily. The total duration of colchicine treatment may vary depending on the situation. For example, the total duration of treatment could be at least 7 days or at least 14 days. The treatment may be repeated for multiple courses depending on the situation; for example, a 30-day course could be repeated three times.

This treatment could be administered in an intermittent fashion. That is, the treatment could be administered for a duration, discontinued for another duration, and then resumed. For example, the treatment could be administered for 2-6 weeks duration, then discontinued for 2-6 weeks duration, and then resumed.

Pyridoxine. The treatment method could further comprise administering pyridoxine (a form of vitamin B6) in combination with the colchicine. The pyridoxine could be administered by any suitable therapeutic route, including oral, intravenous, intramuscular, transdermal, etc. In some cases, the pyridoxine is administered via the same route as the colchicine. The pyridoxine could be administered separately or together (e.g. simultaneously) with the colchicine.

Pyridoxine could enhance the therapeutic efficacy of colchicine. Pyridoxine inhibits many of the same inflammatory cytokines as colchicine. Thus, pyridoxine may work synergistically with the colchicine to enhance the therapeutic effectiveness (e.g. anti-inflammatory action) against the connective tissue disorder. Additionally, pyridoxine can support nerve health in the context of the connective tissue disorder and help manage associated neuropathic symptoms. Pyridoxine may suppress inflammatory responses and support neuropathic recovery, potentially aiding in nerve regeneration and normalizing metabolic processes that are essential for nerve health. As such, pyridoxine may be particularly useful in inflammatory or fibrotic conditions that result in neuropathy. Moreover, the reduction of pain associated with this treatment may further enable increased physical activity, which facilitates proper collagen alignment and promotes extracellular matrix turnover, which thereby improves tissue health and function.

As used herein, the term “pyridoxine” encompasses any pharmaceutical salt form of pyridoxine such as pyridoxine hydrochloride. The pyridoxine is administered at a therapeutically effective dosage amount that enhances the effectiveness of colchicine against the connective tissue disorder. In some embodiments, the route of administration is oral and the dose amount of pyridoxine is in the range of 20-300 mg; and in some cases, 30-250 mg. A single dose or multiple (two or more) doses may be given daily. The pyridoxine used in this invention could be in any suitable pharmaceutical salt form (e.g. pyridoxine hydrochloride).

SOLID FORMULATION. In another aspect, this invention is a solid oral dosage formulation comprising colchicine and pyridoxine. As used herein, “solid oral dosage form” means any orally ingestible form for drug administration having a solid component. Examples include tablets, capsules, powders, sachets, and the like. This combination could be used for treating a connective tissue disorder as described above, or any other suitable condition including gout, and those in oncology, immunology, cardiology, or dermatology.

Colchicine and pyridoxine both possess physiochemical and pharmacokinetic characteristics that suggest good compatibility without significant interaction or interference. Examples of such physiochemical characteristics include lack of highly reactive groups, melting point>40° C., and stability at room temperature. Examples of such pharmacokinetic characteristics include robust water solubility indicating rapid dissolution in the gastrointestinal tract, similar Tₘₐₓ times (pharmacokinetic parameter) after oral ingestion, and distinct, non-competing absorption pathways. Both colchicine and pyridoxine are metabolized and are mostly excreted via renal clearance. We believe that there will be no significant interaction between the metabolites. We also believe that there will be no significant interaction of the drugs or their metabolites during renal clearance. There is no contra-indication against prescribing both drugs together.

The solid oral dosage form contains amounts of colchicine and pyridoxine that are therapeutically effective in synergistic combination. The amount of colchicine contained in the solid dosage form could be 0.2-1.9 mg; and in some cases, 0.5-1.5 mg. The amount of pyridoxine contained in the solid dosage form could be in the range of 20-300 mg; and in some cases, 30-250 mg. The amount of colchicine, pyridoxine, or both could be less than the amount conventionally used singly for achieving the relevant therapeutic effect.

The solid oral dosage form could further contain one or more excipient ingredients, which could function as bulking agents, disintegrants, lubricants for wet granulation, etc. Examples of excipient ingredients that could be used include carnauba wax, hypromellose, polydextrose, polyethylene glycol, triacetin, cellulose gel, croscarmellose sodium, maltodextrin, pre-gelatinized starch, microcrystalline cellulose, sodium starch glycolate, magnesium stearate, and lactose monohydrate

LIQUID FORMULATION. In another aspect, this invention is a liquid oral dosage formulation comprising colchicine and pyridoxine. As used herein, “liquid oral dosage form” means any ingestible liquid drug mixture for oral administration. Examples of such include syrups, elixirs, solutions, suspensions, emulsions, etc. This combination could be used for treating a connective tissue disorder as described above, or any other suitable condition including gout, and those in oncology, immunology, cardiology, or dermatology. As explained above, colchicine and pyridoxine both possess physiochemical and pharmacokinetic characteristics that suggest good compatibility without significant interaction or interference.

The liquid oral dosage form contains amounts of colchicine and pyridoxine that are therapeutically effective in synergistic combination. Expressed as weight amount, the liquid dosage form could contain colchicine in an amount of 0.2-1.9 mg per dose volume; and in some cases, 0.5-1.5 mg. Expressed as weight amount, the liquid dosage form could contain pyridoxine in an amount of 20-300 mg per dose volume; and in some cases, 30-250 mg.

The liquid dosage form could be administered orally in any suitable manner. For example, the liquid could be administered by a drinking cup, spoon, syringe, or medicine dropper. The dose volume of the liquid dosage form could be less than 15 ml or less than 12 ml or less than 7 ml. Expressed as concentration amount, the liquid dosage form could contain colchicine in an amount of 0.02-0.65 mg/ml; and in some cases, 0.05-0.5 mg/ml. Expressed as concentration amount, the liquid dosage form could contain pyridoxine in an amount of 2-100 mg/ml; and in some cases, 3-83 mg/ml. The amount of colchicine, pyridoxine, or both could be less than the amount conventionally used singly for achieving the relevant therapeutic effect.

### DETAILED DESCRIPTION OF EXAMPLE EMBODIMENTS

This invention treats connective tissue disorders. For better understanding, the following is a classification of the various connective tissue disorders that could be treated: tendinopathy (such as tendinosis, tendonitis, or tenosynovitis), nerve compression conditions (such as carpal or tarsal tunnel syndrome), pathologic angiogenesis within connective tissue (such as angiogenesis that causes pain in tendinopathy), and repetitive use injury (e.g. repetitive strain injury or stress injury).

Note that the above categorization is not exclusive or exhaustive. A particular condition may overlap or extend across different categories because it is a primary cause, or a manifestation of another underlying condition, a proximate cause (upstream) thereof, or a downstream effect thereof. All such alternate categorizations would be considered a connective tissue disorder that could be treated by this invention.

Consider for example the context of carpal tunnel syndrome, which could have two or more classifications. Carpal tunnel syndrome could be considered primarily as inflammation of the transverse carpal ligament or the flexor tendons in the wrist that causes compression neuropathy of the median nerve; or it could be considered a manifestation of repetitive strain injury of the wrist.

Or from the alternate viewpoint of wrist tendonitis, repetitive strain injury may be the proximate cause (upstream) of carpal tunnel inflammation or pathologic fibrosis (e.g. with wrist tendon inflammation and swelling thereof leading to compression of the median nerve); whereas compression pinching of the median nerve (causing symptoms of carpal tunnel syndrome) can be considered a downstream effect. Whatever the case may be, reducing inflammation or fibrosis at the carpal tunnel can reduce compression against the median nerve and relieve symptoms of carpal tunnel syndrome. Therefore, in the context of carpal tunnel syndrome, all such associated conditions are encompassed as connective tissue disorders for treatment by this invention.

In some embodiments, the connective tissue disorder is an inflammatory condition. One particular example of a relevant inflammatory condition is tendonitis (inflammation in a tendon), such as Achilles tendonitis, medial epicondylitis (golfer's elbow), lateral epicondylitis (tennis elbow), patellar tendonitis, or wrist tendonitis. In some cases, the connective tissue disorder is not associated with calcium deposits on the connective tissue that are visible on x-rays. In some cases, the connective tissue disorder is not fluoroquinolone-induced tendinopathy. In some cases, the connective tissue disorder is not tenosynovitis.

Colchicine is a medication primarily used to treat gout. It works by disrupting the formation of microtubules in cells. Microtubules are essential components of the cytoskeleton, which is crucial for various cellular processes including cell division and the transport of materials within cells. By inhibiting microtubule polymerization, colchicine impairs the ability of white blood cells to migrate and perform their inflammatory functions. This reduces the inflammatory response and helps alleviate pain and swelling associated with gout.

There are various possible mechanisms and effects of the treatment method described herein. As noted above, colchicine is known to have inhibitory effects against inflammation. Colchicine also has inhibitory effects on fibrosis and angiogenesis. Regarding pathologic fibrosis, this treatment method could regulate the collagenous healing process by modulating (i.e. increasing or decreasing) collagen synthesis. Modulating the amount of collagen in the healing tissue could reduce the buildup of fibrotic tissue and tame overly exuberant tissue growth. Modulating the amount of collagen in the healing tissue could also improve the healing process with the promotion of type I collagen.

The modulation of collagen in the healing tissue could also limit the structural capacity for angiogenesis. Thus, this treatment could have dual anti-angiogenic effects. By this same anti-angiogenic effect, this treatment method could also suppress new nerve generation that leads to pathologic innervation in the tissue. Thus, this treatment could provide a multiprong therapeutic approach that targets the interplay of inflammation, fibrosis, angiogenesis, swelling, or other abnormal tissue remodeling that occurs in connective tissue disorders. This is a significant improvement over conventional therapies that are only effective at symptom relief.

In the context of tendinopathy, the treatment could be based on the patient's stage thereof. In particular, tendinopathy can have three stages: first reactive tendinopathy, then tendon disrepair, and then degenerative tendinopathy. Administering the treatment after the reactive tendinopathy stage may be the optimal time for reducing collagen synthesis. Note also that tendonitis is characterized by tendon inflammation, whereas tendinosis is characterized by tendon degradation. As such, the treatment could treat tendinopathy by acting against inflammation, fibrosis, angiogenesis, or a combination thereof.

The treatment could have various beneficial effects on the pathophysiology of tendinopathy. Tendinopathy may be characterized by an increase in type Ill collagen relative to type I collagen. As such, the treatment may improve the collagen composition (e.g. decrease the ratio of type III collagen relative to type I collagen). Other possible beneficial effects on the pathophysiology of tendinopathy include reducing fibrosis, increasing collagen turnover (e.g. by modulating collagen synthesis or increasing collagenase enzyme activity), increasing extracellular matrix turnover, reducing inflammation, reducing angiogenesis, etc.

COLCHICINE DERIVATIVES. Instead of colchicine, this invention could use chemical derivatives of colchicine. Such colchicine derivatives may be useful in specific medical applications such as the treatment of cancer. See Krzywik et al, “New Series of Double-Modified Colchicine Derivatives: Synthesis, Cytotoxic Effect and Molecular Docking” (2020) Molecules 25, 3540. Many of these derivatives may possess physiochemical properties that are similar to colchicine and thus are amenable to the same dosage forms. Similarly, it is possible to change the physiochemical properties of colchicine by changing the solid-state form. See U.S. Pat. No. 8,309,764 B2 (issued Nov. 13, 2012) titled “Colchicine Solid-State Forms: Methods of Making and Methods of Use Thereof”. Again, many of these modified solid-state forms may possess physiochemical properties that are similar to colchicine, and thus are amenable to the same dosage forms.

The selected chemical derivative of colchicine could be a metabolite of colchicine. Colchicine has two primary metabolites; 2-O-demethylcolchicine and 3-O-demethylcolchicine (2-and 3-DMC, respectively), and one minor metabolite, 10-O-demethylcolchicine.

### Experimental Work (Clinical)

An experimental trial was conducted on a patient, who was a 19 year old man with bilateral upper extremity tendonitis for 7 months duration. The symptoms at the wrist radiated down to his fingers and up to his elbow. He reported the severity of his symptoms as 5-7 on a 1-10 standard discomfort scale. The cause of his widespread tendonitis was repetitive strain injury.

The patient began treatment with 0.6 mg of colchicine by oral intake once daily. By the second day of treatment, symptoms began to improve. With continued daily dosing of colchicine for a total of two weeks duration, symptoms were nearly gone. He was free of symptoms while performing his activities of daily living. At the end of two weeks of treatment, the patient reported reduced severity of symptoms to a level of 2 on a standard 1-10 discomfort scale.

The experimental trial was further extended to observe the effect of discontinuing the colchicine after initially achieving therapeutic success. Upon discontinuing, his tendonitis symptoms gradually returned. After 7 days without treatment, he reported the severity of his symptoms had worsened to 5 on the standard scale. After another 14 days without treatment (21 days cumulative), his symptoms remained at level 5, and occasionally rising to level 6 with increased arm use activity.

### Experimental Work (In Vitro)

In Vitro Tendinopathy Model: The below experiment was conducted on an in vitro tendinopathy model. Cells derived from human tendons (tenocytes) can serve as an in vitro model of tendons. See Miller et al., “MicroRNA29a regulates IL-33-mediated tissue remodelling in tendon disease.” Nat Commun 6, 6774 (2015). Cultured tenocytes can be used to investigate the effects of pro-inflammatory cytokines upon the most basic elements of tendon structure. See Ellis et al., “Defining the profile: Characterizing cytokines in tendon injury to improve clinical therapy.” Journal of Immunology and Regenerative Medicine 16, 100059 (2022). When a tendon is injured, it triggers a local inflammatory response that involves immune cell infiltration and the expression of pro-inflammatory mediators. This inflammatory environment can modify tenocyte physiology, causing them to become activated and pro-inflammatory. Exposing the tenocytes to the pro-inflammatory cytokines TNF-α and IL-1β can mimic the effects of tendon injury. See Smith et al., “Tumour necrosis factor alpha, interleukin 1 beta and interferon gamma have detrimental effects on equine tenocytes that cannot be rescued by IL-1RA or mesenchymal stromal cell-derived factors.” Cell Tissue Res 391, 523-544 (2023).

Cell Culture System. Immortalized, healthy, commercially-sourced human tenocyte cells (58-year-old female donor, patellar tendon) were reconstituted, seeded in T-75 flasks coated with bovine collagen I, and were maintained in Tenocyte Growth Medium supplemented with Tenocyte Growth Supplement, FBS, and penicillin/streptomycin. Culture conditions were maintained at 37° C. in a humidified 5% CO₂ atmosphere. Cells were seeded at a density of 100,000 cells/well in commercially-sourced 24-well plates that were pre-coated with rat tail tendon collagen I.

Treatment Protocol. This study was designed to follow the time-course of collagen production after cytokine exposure to mimic the effects of tendon injury. The time variable was cytokine exposure time (6 hours, 1 day, 3 days, and 5 days). Each of these cytokine exposure time periods was followed by a 24-hour drug treatment period. Inflammatory conditions were induced using a combination of IL-1β (1 nM; 17 ng/ml) and TNF-α (10 ng/ml). Two therapeutic interventions were evaluated. The treatment designated as “treatment A” was colchicine (2 nM). The treatment designated as “treatment B” was colchicine (2 nM) plus pyridoxine (100 μM).

Experimental Groups. The treatment groups were as follows: (1) exposed to inflammatory cytokines plus treatment A; (2) exposed to inflammatory cytokines plus treatment B; (3) exposed to inflammatory cytokines (negative control, no drug). Further reference control groups were as follows: (A) treatment A without inflammatory cytokine exposure (drug-only control); (B) treatment B without inflammatory cytokine exposure (drug-only control); (C) healthy control (no inflammatory cytokines, no drug treatment).

Sample Collection Times. Samples were collected at the following timepoints after initiation of cytokine exposure:
6-hour samples: Collection at 30 hours (6 hr cytokine exposure+24 hr drug treatment)
1-day samples: Collection at 48 hours (24 hr cytokine exposure+24 hr drug treatment)
3-day samples: Collection at 96 hours (72 hr cytokine exposure+24 hr drug treatment)
5-day samples: Collection at 144 hours (120 hr cytokine exposure+24 hr drug treatment, cell culture medium replenished on day-3)

Assays Used. Collagen I (human) content was measured using enzyme-linked immunosorbent assay (ELISA) in cell culture supernatants. TGF-β1 in cell culture supernatants were measured using the MSD multiplex platform. Other inflammatory cytokines in cell culture supernatants were measured using the MSD multiplex platform.

### Collagen I Results

Controls. Healthy control tenocytes demonstrated baseline collagen I production of 30 ng/ml. Treatment A alone, without inflammatory stimulation by cytokines, showed collagen I levels of 17 ng/ml. Negative controls (exposure to inflammatory cytokines, but no drug treatment) were measured at each timepoint.

Six-Hour Collagen Response. The results given below are expressed as concentration levels of human collagen I in the cell culture supernatant. Following 6 hours of cytokine exposure, negative controls showed modestly reduced collagen I levels (at 24 ng/mL) compared to healthy control. Both treatments A and B resulted in similar collagen I levels (at 20 ng/mL for both).

One-Day Collagen Response. At day 1, cytokine-exposed negative controls maintained reduced collagen I levels to 20 ng/ml. Both treatment groups showed comparable collagen I levels: treatment A at 19 ng/ml and treatment B at 19 ng/mL.

Three-Day Collagen Response. At day 3, cytokine-exposed negative controls showed partial recovery of collagen I to 23 ng/ml. Treatment A maintained collagen I levels at 21 ng/ml.

Five-Day Collagen Response. At day 5, cytokine-exposed negative controls showed elevated collagen I levels to 28 ng/ml. Treatment A maintained similar levels (28 ng/mL), while treatment B resulted in moderately lower levels at 22 ng/ml.

Summary of Results. These data demonstrate that both treatment A (colchicine) and treatment B (colchicine+pyridoxine) maintain stable collagen I production under inflammatory conditions. Cytokine exposure causes an initial inflammatory response, which is followed by compensatory elevation in collagen I production with drug treatment. Both treatments A and B showed capability in maintaining collagen I levels within a physiological range, with treatment B (drug combination of colchicine and pyridoxine) demonstrating more consistent measurements at early timepoints. These results suggest that colchicine (with and without pyridoxine) will sustain collagen I production under inflammatory conditions of tendon injury.

### TGF-β1 Results

Controls. Healthy control tenocytes demonstrated baseline TGF-β1 production of 1.73 ng/ml. Treatment A alone, without inflammatory stimulation by cytokines, showed TGF-β1 levels of 1.90 ng/ml. Negative controls (exposure to inflammatory cytokines, but no drug treatment) were measured at each timepoint.

Six-Hour Response. The results given below are expressed as concentration levels of TGF-β1 in the cell culture supernatant. Following 6 hours of cytokine exposure, negative controls showed substantially reduced TGF-β1 levels (to 0.97 ng/ml) compared to healthy control. Treatment A resulted in elevated TGF-β1 levels of 1.23 ng/ml (127% of negative control), while treatment B showed similar improvement to 1.16 ng/ml (120% of negative control).

One-Day Response. At day 1, cytokine-exposed negative controls maintained reduced TGF-β1 levels at 1.22 ng/ml (71% of healthy baseline). Both treatment groups showed improvement in TGF-β1 levels: treatment A at 1.28 ng/ml (104% of negative control) and treatment B at 1.43 ng/ml (117% of negative control). Drug-only control showed enhanced TGF-β1 production at 1.90 ng/ml (110% of healthy baseline).

Three-Day Response. At day 3, cytokine-exposed negative controls showed recovery of TGF-β1 to 1.70 ng/ml (98% of healthy baseline). Treatment A maintained elevated TGF-β1 levels at 1.94 ng/ml (114% of negative control, 112% of healthy baseline).

Five-Day Response. At day 5, cytokine-exposed negative controls showed renewed suppression of TGF-β1 to 1.22 ng/mL (71% of healthy baseline). Treatment A maintained higher levels at 1.32 ng/ml (108% of negative control), while treatment B demonstrated superior response at 1.52 ng/ml (124% of negative control, 88% of healthy baseline).

Summary of Results. These data demonstrate that both treatment A (colchicine) and treatment B (colchicine+pyridoxine) effectively counteract cytokine-induced TGF-β1 suppression. The inflammatory response causes an initial reduction in TGF-β1 levels to approximately 56% of healthy baseline at 6 hours, with sustained suppression through day 5, except for a temporary recovery at day 3. Both treatments show capability in elevating TGF-β1 levels above negative control values, with treatment B demonstrating superior sustained effects by day 5. Treatment A showed particularly strong effects at day 3 (114% of negative control) before moderating by day 5. These results suggest that colchicine, particularly in combination with pyridoxine, effectively maintains TGF-β1 production under inflammatory conditions of tendon injury.

### IFN-γ Results

Controls. For the healthy control sample, IFN-γ level was 8.2 pg/mL. Treatment A alone, without inflammatory stimulation by cytokines, demonstrated IFN-γ levels of 3.1 pg/mL. Negative controls (exposure to inflammatory cytokines, but no drug treatment) were measured at each timepoint.

Six-Hour Response. The results given below are expressed as concentration levels of IFN-γ in the cell culture supernatant. Following 6 hours of cytokine exposure, negative controls showed markedly elevated IFN-γ levels at 17.6 pg/ml (215.7% of healthy level). Treatment A demonstrated significant reduction to 12.6 pg/ml (71.6% of negative control, 154.5% of healthy level), while treatment B resulted in further elevation to 21.4 pg/ml (121.4% of negative control, 261.8% of healthy level).

One-Day Response. At day 1, cytokine-exposed negative controls maintained elevated IFN-γ levels at 15.6 pg/ml (190.7% of healthy level). Treatment A showed modest reduction to 15.3 pg/mL (98.0% of negative control, 186.9% of healthy level), while treatment B resulted in further elevation to 19.9 pg/ml (127.6% of negative control, 243.4% of healthy level).

Three-Day Response. At day 3, cytokine-exposed negative controls showed sustained elevation of IFN-γ at 12.7 pg/mL (154.8% of healthy level). Treatment A reduced IFN-γ levels to 9.2 pg/mL (72.4% of negative control, 112.1% of healthy level).

Five-Day Response. At day 5, cytokine-exposed negative controls showed continued elevation of IFN-γ at 16.3 pg/mL (199.0% of healthy level). Treatment A demonstrated modest reduction to 13.1 pg/mL (80.7% of negative control, 160.7% of healthy level), while treatment B maintained levels at 15.2 pg/ml (93.2% of negative control, 185.4% of healthy level).

Summary of Results. These data demonstrate that inflammatory cytokine exposure results in sustained elevation of IFN-γ levels to approximately 170-190% of healthy baseline. Treatment A (colchicine) shows early efficacy in opposing this elevation, particularly at the 6-hour timepoint where it reduces IFN-γ levels by 28.4% compared to negative control. This opposing effect is maintained through day 5, albeit at reduced magnitude. Treatment B (colchicine+pyridoxine) demonstrates a contrasting response pattern, generally maintaining or increasing IFN-γ elevation. These results suggest that colchicine monotherapy may be more effective than the combination treatment for modulating IFN-γ responses in inflammatory conditions.

### IL-6 Results

Controls. For the healthy control sample, IL-6 level was 342.3 pg/mL. Treatment A alone, without inflammatory stimulation by cytokines, demonstrated reduced IL-6 levels of 73.7 pg/mL. Negative controls (exposure to inflammatory cytokines, but no drug treatment) were measured at each timepoint.

Six-Hour Response. The results given below are expressed as concentration levels of IL-6 in the cell culture supernatant. Following 6 hours of cytokine exposure, negative controls showed elevation of IL-6 levels at 1614.8 pg/mL (471.7% of healthy level). Treatment A demonstrated substantial elevation to 2989.5 pg/ml (185.1% of negative control, 873.4% of healthy level), while treatment B resulted in moderate elevation to 2239.1 pg/ml (138.7% of negative control, 654.1% of healthy level).

One-Day Response. At day 1, cytokine-exposed negative controls maintained elevated IL-6 levels at 1726.6 pg/mL (504.4% of healthy level). Treatment A showed further elevation to 2114.9 pg/mL (122.5% of negative control, 617.8% of healthy level), while treatment B demonstrated reduction to 1543.9 pg/ml (89.4% of negative control, 451.0% of healthy level).

Three-Day Response. At day 3, cytokine-exposed negative controls showed reduction in IL-6 to 1067.0 pg/mL (311.7% of healthy level). Treatment A demonstrated significant elevation to 1360.1 pg/mL (127.5% of negative control, 397.3% of healthy level), opposing the cytokine-induced reduction.

Five-Day Response. At day 5, cytokine-exposed negative controls maintained reduced IL-6 levels at 696.6 pg/mL (203.5% of healthy level). Treatment A showed continued opposing effect with elevation to 708.5 pg/ml (101.7% of negative control, 207.0% of healthy level), while treatment B maintained levels at 938.0 pg/ml (134.7% of negative control, 274.0% of healthy level).

Summary of Results. These data demonstrate that inflammatory cytokine exposure produces a biphasic IL-6 response, with initial elevation followed by sustained reduction. Treatment A (colchicine) shows consistent modulatory effects throughout the time course, with particularly significant opposing effects during the late phase (days 3-5) where it partially restores IL-6 levels toward baseline. Treatment B (colchicine+pyridoxine) demonstrates more modest effects, suggesting potential mechanistic differences between colchicine monotherapy and combination treatment in IL-6 regulation.

### TNF-α Results

Controls. For the healthy control sample, TNF-α level was 211.3 pg/mL. Treatment A alone, without inflammatory stimulation by cytokines, demonstrated minimal TNF-α levels of 1.1 pg/mL. Negative controls (exposure to inflammatory cytokines, but no drug treatment) were measured at each timepoint.

Six-Hour Response. The results given below are expressed as concentration levels of TNF-α in the cell culture supernatant. Following 6 hours of cytokine exposure, negative controls showed marked elevation of TNF-α levels to 1678.4 pg/mL (794.4% of healthy level). Treatment A resulted in further elevation to 1958.4 pg/ml (116.7% of negative control, 926.9% of healthy level), while treatment B showed modest elevation to 1782.0 pg/ml (106.2% of negative control, 843.4% of healthy level).

One-Day Response. At day 1, cytokine-exposed negative controls remained elevated at 1317.2 pg/mL (623.4% of healthy level). Treatment A demonstrated elevation to 1703.5 pg/ml (129.3% of negative control, 806.3% of healthy level), while treatment B showed modest increase to 1460.3 pg/mL (110.9% of negative control, 691.1% of healthy level).

Three-Day Response. At day 3, cytokine-exposed negative controls showed reduction in TNF-α to 960.1 pg/mL (454.4% of healthy level). Treatment A elevated TNF-α levels to 1303.6 pg/mL (135.8% of negative control, 617.0% of healthy level).

Five-Day Response. At day 5, cytokine-exposed negative controls showed sustained reduction of TNF-α to 824.1 pg/mL (390.1% of healthy level). Treatment A demonstrated significant elevation to 895.9 pg/ml (108.7% of negative control, 424.1% of healthy level), while treatment B maintained levels at 1036.5 pg/mL (125.8% of negative control, 490.6% of healthy level).

Summary of Results. These data demonstrate that inflammatory cytokine exposure results in a complex TNF-α response pattern, with initial elevation followed by progressive reduction. Treatment A (colchicine) shows consistent modulatory effects throughout the time course, with particularly notable opposing effects during the late phase where it partially restores TNF-α levels toward baseline. Treatment B (colchicine+pyridoxine) demonstrates more modest effects, suggesting a mechanism of action in TNF-α regulation that is distinct from treatment A.

### IL-33 Results

Controls. For the healthy control sample, IL-33 level was 8.7 pg/mL. Treatment A alone, without inflammatory stimulation by cytokines, demonstrated reduced IL-33 levels of 3.4 pg/mL. Negative controls (exposure to inflammatory cytokines, but no drug treatment) were measured at each timepoint.

Six-Hour Response. The results given below are expressed as concentration levels of IL-33 in the cell culture supernatant. Following 6 hours of cytokine exposure, negative controls showed substantial elevation of IL-33 levels to 18.0 pg/mL (206.6% of healthy level). Treatment A demonstrated significant reduction to 9.9 pg/mL (55.0% of negative control, 113.6% of healthy level), while treatment B maintained elevated levels at 16.7 pg/ml (92.8% of negative control, 191.7% of healthy level).

One-Day Response. At day 1, cytokine-exposed negative controls maintained elevated IL-33 levels at 13.1 pg/mL (150.6% of healthy level). Treatment A showed modest reduction to 11.2 pg/mL (85.1% of negative control, 128.1% of healthy level), while treatment B resulted in further elevation to 19.4 pg/ml (147.4% of negative control, 221.9% of healthy level).

Three-Day Response. At day 3, cytokine-exposed negative controls showed sustained elevation of IL-33 at 10.7 pg/mL (123.1% of healthy level). Treatment A demonstrated continued opposing effect with reduction to 7.6 pg/ml (70.8% of negative control, 87.1% of healthy level).

Five-Day Response. At day 5, cytokine-exposed negative controls maintained elevated IL-33 levels at 17.1 pg/ml (195.9% of healthy level). Treatment A showed persistent opposing effect with reduction to 14.6 pg/mL (85.4% of negative control, 167.4% of healthy level), while treatment B maintained levels at 13.0 pg/ml (76.3% of negative control, 149.4% of healthy level).

Summary of Results. These data demonstrate that inflammatory cytokine exposure results in sustained elevation of IL-33 levels throughout the experimental time course. Treatment A (colchicine) shows consistent opposing effects (i.e. reduction in IL-33) at all timepoints, with particularly significant reduction at 6 hours where it reduces IL-33 levels by 45% compared to negative control. This opposing effect is maintained through day 5, demonstrating sustained therapeutic activity. Treatment B (colchicine+pyridoxine) shows minimal regulatory effect on IL-33 levels, suggesting that the colchicine monotherapy may be more effective for IL-33 modulation than the combination therapy.

### VEGF Results

Controls. For the healthy control sample, VEGF level was 199.0 pg/mL. Treatment A alone, without inflammatory stimulation by cytokines, demonstrated elevated VEGF levels of 621.7 pg/mL. Negative controls (exposure to inflammatory cytokines, but no drug treatment) were measured at each timepoint.

Six-Hour Response. The results given below are expressed as concentration levels of VEGF in the cell culture supernatant. Following 6 hours of cytokine exposure, negative controls showed elevation of VEGF levels to 333.6 pg/mL (167.6% of healthy level). Treatment A demonstrated significant elevation to 523.8 pg/ml (157.0% of negative control, 263.2% of healthy level), while treatment B showed similar elevation to 476.4 pg/ml (142.8% of negative control, 239.4% of healthy level).

One-Day Response. At day 1, cytokine-exposed negative controls showed elevated VEGF levels at 599.5 pg/mL (301.2% of healthy level). Treatment A resulted in elevation to 799.7 pg/ml (133.4% of negative control, 401.8% of healthy level), while treatment B showed reduction to 455.4 pg/mL (76.0% of negative control, 228.8% of healthy level).

Three-Day Response. At day 3, cytokine-exposed negative controls showed elevation in VEGF to 555.0 pg/mL (278.9% of healthy level). Treatment A demonstrated elevation to 624.9 pg/ml (112.6% of negative control, 314.0% of healthy level).

Five-Day Response. At day 5, cytokine-exposed negative controls showed sustained elevation of VEGF to 386.6 pg/mL (194.2% of healthy level). Treatment A maintained elevation to 455.8 pg/ml (117.9% of negative control, 229.0% of healthy level), while treatment B showed levels at 518.8 pg/mL (134.2% of negative control, 260.7% of healthy level).

Summary of Results. These data demonstrate that inflammatory cytokine exposure results in sustained elevation of VEGF levels throughout most of the experimental time course. Treatment A (colchicine) shows consistent elevating effects on VEGF at all timepoints, with particularly significant elevation at 6 hours where it increases VEGF levels by 57% compared to negative control. This effect is maintained through day 5, demonstrating sustained activity. Treatment B (colchicine+pyridoxine) shows early elevating effects similar to colchicine monotherapy but variable activity at later timepoints.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of treating a connective tissue disorder in a patient, comprising orally administering colchicine in a dose amount in the range of 0.2-1.9 mg.

2. The method of claim 1, wherein the method treats a target connective tissue, wherein the target connective tissue is a tendon, ligament, tendon sheath, or subsynovial connective tissue.

3. The method of claim 1, wherein the connective tissue disorder is tendinopathy.

4. The method of claim 3, wherein the tendinopathy is carpal tunnel syndrome.

5. The method of claim 1, wherein the dose amount is in the range of 0.5-1.5 mg.

6. The method of claim 2, wherein the treatment reduces fibrosis in the target connective tissue.

7. The method of claim 2, wherein the treatment promotes collagen turnover in the target connective tissue.

8. The method of claim 1, wherein pyridoxine is not administered to the patient together with the colchicine.

9. The method of claim 2, wherein the treatment increases the ratio of type I collagen to type III collagen.

10. The method of claim 2, wherein the treatment promotes extracellular matrix turnover in the target connective tissue.

11. The method of claim 2, wherein the treatment reduces inflammation in the target connective tissue.

12. The method of claim 1, wherein the treatment reduces angiogenesis in the target connective tissue.

13. The method of claim 1, wherein the colchicine is administered multiple times daily.

14. The method of claim 1, wherein the duration of treatment is at least 7 days.

15. The method of claim 14, wherein the duration of treatment is at least 14 days. 16 The method of claim 1, further comprising administering pyridoxine in combination with the colchicine.

17. The method of claim 16, wherein the pyridoxine is administered in a dose amount in the range of 20-300 mg.

18. The method of claim 2, wherein the target connective tissue does not have calcium deposits that are visible on x-ray.

19. The method of claim 1, wherein the connective tissue disorder is not fluoroquinolone-induced tendinopathy.

20. The method of claim 1, wherein the connective tissue disorder is not tenosynovitis.
